(19) United States
(12) Patent Application Publication
(10) Pub. No.: US 2013/0252875 A1
(43) Pub. Date: Sep. 26, 2013

(54) Thickener Systems For Personal Care and Other Cleansing Compositions

(71) Applicant: Alla Merces, Hillsborough, NC (US)

(72) Inventor: Alla Merces, Hillsborough, NC (US)

(21) Appl. No.: 13/428,459

(22) Filed: Mar. 23, 2012

(51) Int. Cl. A61K 8/73; A61Q 5/02; A61Q 19/10

(52) U.S. Cl. 510/405; 510/513

(57) ABSTRACT

Disclosed herein are liquid personal care cleansing compositions comprising a carrier (e.g., aqueous), a detergent surfactant for personal cleansing (i.e., hair, skin, or nails), and a thickening system that may include components that are naturally-derived and naturally-processed. The thickening system includes a gum, a lactate, and a lactylate, and is substantially free of synthetic amides such as ethanolamines. The lactate and lactylate have surprisingly been found to break the gelatinous, gooey blob characteristics that result when thickening with a natural gum alone, resulting in a homogenous smooth consistency to the personal care composition thickened with the thickener system.

### FIELD OF THE INVENTION

Disclosed herein are formulations related to personal care cleansing compositions suitable for topical application for cleansing skin and hair. More particularly, disclosed herein are thickening systems for such compositions. The compositions may be produced from a limited number of naturally-derived and naturally-processed ingredients. The compositions provide thickening or consistency characteristics similar to those achieved with synthetic thickening systems.

### DESCRIPTION OF RELATED ART

Personal care cleansing compositions have progressed and supported a large chemical industry devoted to developing new synthetic surfactants and moisturizers and other components to achieve ever improving personal care cleansers for the consumer. There are currently over ten thousand different synthetic chemicals used in personal care products intended for use on human skin. Although these synthetic chemicals have been tested on animals at some stage, they have never been tested for long term health affects either individually or in combination. Even the majority of those that actually have been found to cause an adverse health effect to some members of the population can still be used.

Typical personal cleansing formulations require multiple synthetic ingredients many of which have suspected human health risks as indicated by peer-reviewed third-party scientific literature. For example, the following synthetic ingredients are commonly found in personal care cleansing formulations: parabens; chemical sunscreens such as avobezone and oxybenzone; glycols; phthalates; and ethoxylated ingredients such as sodium myreth sulfate, sodium laureth sulfate, PEGs (polyethylene glycol) and PPGs (polypropylene glycol); ethanolamines such as DEAs (diethanolamine), MEAs (monoethanolamine), TEAs (triethanolamine); synthetic polymers such as PVPs (polyvinylpyrrolidone) and acrylates; and formaldehyde donors such as DMDM hydantoin, diazolidinyl urea and methylisothiazolinone.

In addition to numerous synthetic ingredients, many personal care cleansing formulations may have otherwise natural ingredients that are synthetically-derived or processed. Processes such as ethoxylation, sulfination or polymerization have the potential to change the chemical make-up of ingredients that start out natural, but may not remain so after processing. These types of processes dilute or change the composition of an ingredient and can involve caustic solvents, impurities and leave residual compounds behind. Natural, ecological processes such as distillation, condensation, extraction, steam distillation, pressure cooking and hydrolysis are desirable to maximize the purity of natural ingredients.

Because of a desire to use renewable resources and to eliminate contact with potentially harmful synthetic materials, natural-based personal care cleansing compositions are gaining increasing interest. Most of these personal cleansing compositions contain only some natural ingredients with the majority of their components being synthetic. One difficulty in formulating all-natural personal cleansing compositions is achieving acceptable consumer performance with a limited number of raw materials. The number of all-natural ingredients available is scarce when compared to the number of highly developed synthetic ingredients.

One particular problem when formulating natural personal care cleansing compositions is with achieving a desired level of viscosity (i.e., thickening), while also providing consistency characteristics that are homogenous throughout the bulk of the composition. For example, attempts to use natural gums for thickening natural-based compositions have often resulted in gooey gelatinous blobs within the bulk of the composition rather than a homogenous consistency similar to that easily achieved using synthetic components. It is therefore desirable to provide a personal care cleansing composition that overcomes the disadvantages and shortcomings associated with those of the prior art.

### BRIEF SUMMARY OF THE INVENTION

One aspect of the invention relates to liquid personal care cleansing compositions comprising a carrier (e.g., aqueous), a detergent surfactant for personal cleansing (i.e., hair, skin, or nails), and a novel thickening system including components that may be naturally-derived and naturally-processed. The novel thickening system includes a gum, a lactate, and a lactylate, and is substantially free of amides (e.g., ethanolamines).

In another aspect of the invention, the thickening system is an amide-free, natural thickening system for use in liquid personal care cleansing compositions that comprises components that are naturally-derived and naturally-processed.

In a third aspect of the invention, both the thickener system and the personal care cleansing composition comprise primarily naturally-derived and naturally-processed components. Such compositions are free of ethanolamines and may also be free of sulfates.

Thickener systems used in shampoos and other personal care cleansing products have typically included various ethanolamine components for their excellent thickening ability. While such amine components exhibit excellent ability to thicken liquid compositions, these synthetic materials are not naturally-derived or naturally-processed, and there are suspected health risks associated with their use. As such, a need exists for thickener systems that include components that are naturally-derived and naturally-processed.

Some have attempted to use natural gums for this purpose, although such gums are not able to provide a homogenous consistency throughout the liquid composition they are intended to thicken. For example, the use of natural gums for thickening tends to result in lumpy, gooey, gelatinous blobs within the liquid product composition. Such results are perceived as “cheap”, and are clearly unacceptable to many consumers who have become accustomed to the excellent consistency of thickened compositions achieved with synthetic thickening components.

The present inventor has surprisingly found that when combining a natural gum with a lactate and a lactylate component (each of which may be naturally-derived and naturally-processed), thickening characteristics similar to those provided by synthetic amine components can be achieved.

In some embodiments, the personal cleansing compositions disclosed herein contain at least 95% naturally-derived and naturally-processed components. Unlike prior art “natural” formulations, the compositions disclosed herein have been shown to be as good or better than their synthetic or quasi-synthetic counterparts.

The features and advantages of embodiments of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE PREFERRED EMBODIMENTS

### I. Definitions

Before describing embodiments of the present invention in detail, it is to be understood that they are not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

References herein to “one embodiment”, “one aspect” or “one version” of the invention include one or more such embodiments, aspects or versions, unless the context clearly dictates otherwise.

As used in this specification and the appended claims, the singular forms “a”, “an”, and “the” include plural referents unless the context clearly dictates otherwise.

Unless defined otherwise, all technical and scientific terms used herein have the same meaning as commonly understood by one of ordinary skill in the art to which embodiments of the invention pertain. Although a number of methods and materials similar or equivalent to those described herein can be used in the practice of embodiments of the present invention, the preferred materials and methods are described herein.

In the application, effective amounts are generally those amounts listed as the ranges or levels of ingredients in the descriptions, which follow hereto. Unless otherwise stated, amounts listed in percentage (“%'s”) are in weight percent (based on 100% active) of the active composition alone, unless otherwise indicated.

The term “comprising”, which is synonymous with “including,” “containing,” or “characterized by,” is inclusive or open-ended and does not exclude additional, unrecited elements or method steps. See MPEP 2111.03. See, e.g., Mars Inc. v. H.J. Heinz Co., 377 F.3d 1369, 1376, 71 USPQ2d 1837, 1843 (Fed. Cir. 2004) (“like the term ‘comprising,’ the terms ‘containing’ and ‘mixture’ are open-ended.”). Invitrogen Corp. v. Biocrest Mfg., L.P., 327 F.3d 1364, 1368, 66 USPQ2d 1631, 1634 (Fed. Cir. 2003) (“The transition ‘comprising’ in a method claim indicates that the claim is open-ended and allows for additional steps.”); Genentech, Inc. v. Chiron Corp., 112 F.3d 495, 501, 42 USPQ2d 1608, 1613 (Fed. Cir. 1997) See MPEP 2111.03. (“Comprising” is a term of art used in claim language which means that the named elements are essential, but other elements may be added and still form a construct within the scope of the claim.); Moleculon Research Corp. v. CBS, Inc., 793 F.2d 1261, 229 USPQ 805 (Fed. Cir. 1986); In re Baxter, 656 F.2d 679, 686, 210 USPQ 795, 803 (CCPA 1981); Ex parte Davis, 80 USPQ 448, 450 (Bd. App. 1948). See MPEP 2111.03.

The term “consisting essentially of” as used herein, limits the scope of a claim to the specified materials or steps “and those that do not materially affect the basic and novel characteristic(s)” of the claimed invention. In re Herz, 537 F.2d 549, 551-52, 190 USPQ 461, 463 (CCPA 1976) (emphasis in original). See MPEP 2111.03.

“All numbers expressing quantities of ingredients, constituents, reaction conditions, and so forth used in the specification and claims are to be understood as being modified in all instances by the term “about”. Notwithstanding that the numerical ranges and parameters setting forth the broad scope of the subject matter presented herein are approximations, the numerical values set forth in the specific examples are reported as precisely as possible. Any numerical value, however, inherently contains certain errors necessarily resulting from the standard deviation found in their respective testing measurements.”

The term “personal” or “personal care” as used herein means a composition suitable for use on skin, hair and nails.

The term “naturally-derived” as used herein is meant to mean that the ingredient comes or is made from a renewable resource found in nature (i.e., Flora, Fauna, Mineral). Petroleum compounds are expressly excluded from the term “naturally-derived”.

The term “naturally-processed” as used herein means the ingredients are processed using only ecologically-friendly processes. Ecologically-friendly processing is minimal processing that maximizes purity and minimizes negative effects on the ingredients. Only biodegradable reagents are used in ecologically-friendly processing. Naturally-processed as used herein includes, but is not limited to, distillation, condensation, extraction, steamed distillation, pressure cooking and hydrolysis.

Other examples of natural processes include: saponification which uses a strong alkali base (e.g., NaOH) to create a reaction with a fat or oil to produce soap, glycerin and water in one process; esterification and transesterification which involve reacting an alcohol and an acid or base to create safe emulsifiers, surfactants and solubilizers that thicken, hydrate, and/or moisturize; and biofermentation which converts substances through the use of a yeast and/or a bacteria to produce nutrients and/or to purify formulations. The microorganisms typically do not survive the process.

When a component is described as being naturally-derived and naturally-processed, it is meant that the component is naturally-derived, and if any processing is performed, the processing is natural. Some naturally-derived components may not require any processing, and such components are still described as naturally-derived and naturally-processed.

Several processes dilute or change the composition of an ingredient to the point that an otherwise natural ingredient becomes “unnatural” by virtue of how it is processed. Ethoxylation, sulfonation and polymerization processes are expressly excluded from the term “naturally-processed”. For example, sulfination uses harsh processing that involves sulfates, sulfonates and/or phosphates to create effective wetting agents for use in detergents and foaming agents. Such processes are excluded from the definition of “naturally-processed” because sulfates do not break down in the environment. One example of a naturally-derived synthetic substitute produced by processes such as sulfination and ethoxylation is called sodium lauryl/laureth sulfate (SLS). SLS is derived from coconut oil, but is processed in such a way that does not render it an “eco-friendly, natural” ingredient as the term is used herein.

The term “GRAS” as used herein is meant to mean generally recognized as safe (GRAS) by the Food and Drug Administration (FDA) when used in accordance with the FDA's good manufacturing practices (GMP) and contain no residues of heavy metals or other contaminants in excess of tolerances set by the FDA or the EPA.

The term “biodegradable” as used herein refers to carbon containing materials that can be degraded by microbial action. The “biodegradable” materials may be tested under a recognized protocol and with tested methods of established regulatory bodies such as: EPA, EPA-TSCA, OECD, MITI or other similar or equivalent organizations in the US or internationally. Suitable non-limiting examples of test methods for biodegradation include: OECD methods in the 301-305 series. Generally, all “biodegradable” materials must meet the following limitations:

removal of dissolved organic carbon >70%

biological oxygen demand (BOD)>60%

% of BOD of theoretical oxygen demand >60%

% CO₂ evolution of theoretical >60%

In one embodiment, personal cleansing compositions of the present invention contain at least 95% naturally-derived, naturally-processed components. Unlike prior art thickened “natural” formulations (e.g., those thickened with only natural gums), these embodiments of the present invention have been shown to have thickening characteristics that are as good or better than their synthetic or quasi-synthetic counterparts. Of course, although less preferred, the novel thickening systems disclosed herein could be used in personal care cleansing compositions that contain less than 95% naturally-derived, naturally-processed components.

### II. Introduction

One embodiment of the present invention is directed to thickening systems for use in liquid personal care cleansing compositions. The thickener system is advantageously formed of naturally-derived, naturally-processed components, and includes a natural gum, a lactate, and a lactylate. The system is advantageously free of synthetic amide components (e.g., ethanolamines). The thickener system may be employed in liquid personal care cleansing compositions that also comprise a carrier that is naturally-derived and naturally-processed and a detergent surfactant for personal cleansing. The thickener systems allow thickening of compositions without the need for addition of synthetic amides, while providing homogenous consistency characteristics throughout the bulk of the composition. In some embodiments, the liquid personal care cleansing compositions are free of synthetic amides and may also be free of sulfates.

The present inventor has surprisingly found that although a natural gum alone is not able to provide homogenous thickened consistency throughout a liquid personal care cleansing composition, the addition of both a lactate and a lactylate component breaks the gelatinous, gooey blob characteristics associated with use of natural gums alone. This is further surprising as lactates and lactylates, when used alone or even in combination with one another are not able to provide a viscosity within the desired range (e.g., typically about 1,000 cps to about 10,000 cps or about 2,000 cps to about 10,000 cps). This being said, it has surprisingly been found that the addition of both a lactate and a lactylate to the natural gum breaks the gelatinous, non-homogenous thickening characteristics provided by the gum, resulting in a substantially homogenous thickened composition having thickening characteristics similar to those provided by synthetic ethanolamine components.

Such personal care cleansing compositions may include shampoos, conditioners, facial washes, body washes, liquid soaps, and liquid cleansers. The thickening systems may also find application in liquid laundry compositions, liquid dish soaps, and other liquid cleaning compositions.

### III. Exemplary Thickening Systems and Personal Care Cleansing Compositions

A. Gums

The thickener systems according to the present invention include one or more gum components. Such gums are generally polysaccharides. Such gums may be synthetic or extracted or derived from various natural sources such as marine plants (e.g., seaweed), non-marine plants, bacterial fermentation, or other natural sources. Gums that may be suitable for use that can be naturally derived from seaweed include, but are not limited to, agar, alginic acid, sodium alginate, and carrageenan. Gums that may be suitable for use that can be naturally derived from non-marine botanical sources include, but are not limited to, gum arabic, gum ghatti, gum tragacanth, karaya gum, guar gum, locust bean gum, beta-glucan, chicle gum, dammar gum, glucomannan, mastic gum, psyllium gum (i.e., psyllium seed husks from the Plantago plant), spruce gum, and tara gum. Gums that may be suitable for use that can be naturally produced by bacterial fermentation include, but are not limited to, gellan gum and xanthan gum.

Particularly preferred gums suitable for use in the thickening systems disclosed herein include xanthan gum, carrageenan, and gum arabic. The gum may be included within the personal care cleansing composition in a range of about 0.01% to about 5%, about 0.05% to about 3%, or about 0.1% to about 2% by weight of the personal care cleansing composition.

B. Lactates

The thickener systems according to the present invention include one or more lactate components. Such lactates may generally be conjugate bases of lactic acid (C₃H₆O₃). In one embodiment, the lactate may be the reaction product of an alcohol (e.g., a fatty alcohol) and lactic acid, forming an ester. In one embodiment, such esterification products may include a fatty acid chain having from 4 to 30 carbon atoms, from 6 to 20 carbon atoms, or from 12 to 18 carbon atoms. Various suitable fatty acid chains may be straight, branched, saturated, or unsaturated. Particularly preferred lactates include the reaction products of a fatty acid alcohol comprising 12 to 18 carbon atoms and lactic acid. Lauryl lactyl lactate is one such suitable lactate material, which is the esterification product of lauryl alcohol and lactic acid. Other specific suitable lactates may include, but are not limited to myristyl lactyl lactate, cetyl lactyl lactate, stearyl lactyl lactate, and combinations thereof. Such lactates may be naturally-processed through esterification from naturally-derived reaction components.

The one or more lactate components of the thickener system may be included within the personal care cleansing composition in a range of about 0.01% to about 3%, about 0.05% to about 2%, or about 0.1% to about 1% by weight of the personal care cleansing composition.

C. Lactylates

The thickener systems according to the present invention include one or more lactylate components. The lactylate group is also known as lactic acid dimer, as it can be formed by esterification between two lactic acid molecules. Similar to the lactate, the lactylate may also typically include a fatty acid chain. In one embodiment, the fatty acid chain may include from 4 to 30 carbon atoms, from 6 to 20 carbon atoms, or from 12 to 18 carbon atoms. Various suitable fatty acid chains may be straight, branched, saturated, or unsaturated. Classes of suitable lactylates include alkali metal salt (e.g., sodium or potassium) lactylates and alkaline earth metal (e.g., magnesium or calcium) salt lactylates. One particularly preferred lactylate is sodium lauroyl lactylate. Other specific suitable lactylates may include, but are not limited to sodium stearoyl lactylate, calcium stearoyl lactylate, sodium behenoyl lactylate, sodium caproyl lactylate, sodium caproyl/lauroyl lactylate, sodium cocoyl lactylate, sodium isostearoyl lactylate, sodium lauroyl lactylate, sodium oleoyl lactylate, and combinations thereof. Such lactylates may be naturally-processed through esterification from naturally-derived reaction components.

The one or more lactylate components of the thickener system may be included within the personal care cleansing composition in a range of about 0.1% to about 5%, about 0.25% to about 3%, or about 0.5% to about 2% by weight of the personal care cleansing composition.

As the lactate and lactylate components together serve to break the gelatinous characteristics of the natural gum thickener, the ratio of these various components relative to one another may be an important factor for consideration. In one embodiment, the weight ratio of the gum to the lactylate may be from about 0.1:1 to about 2:1, or from about 0.5:1 to about 1:1. In another embodiment, the weight ratio of the gum to the lactate may be from about 0.1:1 to about 10:1, or from about 2:1 to about 8:1. In a third embodiment, the weight ratio of the lactylate to the lactate may be from about 0.05:1 to about 0.75:1, or from about 0.1:1 to about 0.25:1.

Some embodiments may also include an inorganic salt of an alkali metal or alkaline earth metal and a halogen (e.g., sodium chloride) as part of the thickener system. Such inorganic salts increase viscosity, and may be present in an amount from about 0.01% to about 3%, from about 0.05% to about 2%, or from about 0.1% to about 0.5% by weight of the personal care cleanser composition.

Preferred characteristics of a final formulation include an attractive appearance and color (e.g., creamy white to off-white), a pH in the range of 4.5-6.5, a viscosity in the range of about 1,000 to about 10,000 cps, and specific gravity in the range of 0.990-1.100.

D. Carriers

Liquid personal care cleansing compositions thickened with thickener systems according to the present invention include one or more carrier components. In one embodiment, the principal carrier may be water, such that the carrier is aqueous. Some embodiments may include at least about 50% water (e.g., about 50% to about 80% water). Other carrier components may also be used, such as alcohols. Polyols are a particular class of alcohols that may be particularly useful as carrier components. Examples of such polyols include, but are not limited to, glycerin, polyethylene glycol, propylene glycol, and combinations thereof, although it may be preferable to avoid the use of synthetic polyols such as polyethylene glycol and propylene glycol in at least some embodiments. In one embodiment, any polyol components employed are naturally-derived (e.g., glycerin derived from vegetable oil).

Glycerin is an example of a naturally-derived and naturally-processed polyol carrier exhibiting humectant properties. Examples of other humectant carriers include other polyols, as well as lecithin and honey. Glycerin is a neutral, thick liquid which freezes to a gummy paste and has a high boiling point. Glycerin can be dissolved into water or alcohol, but generally not into oils. In addition, many components can be dissolved into glycerin more easily than into water or alcohol. Thus, glycerin can be used to solubilize other components, including a gum (e.g., xanthan gum) of the thickener system. A humectant carrier (e.g., glycerin) may be included in amounts ranging from about 0.5% to about 20%, from about 1% to about 10%, or from about 1% to about 5% by weight of the personal care cleansing composition.

E. Detergent Surfactants

In embodiments of the present invention, cleansing is provided by detergent surfactants. A detergent surfactant is a surface active agent (i.e., a surfactant) that when dissolved in water is able to emulsify oils and hold dirt or other insoluble foreign matter in suspension. This provides the personal care cleansing product with the ability to remove dirt, sweat, sebum, exfoliated skin tissue, and oils from skin and hair. Such surfactants also enable the product to fully wet the skin and hair so that environmental dirt and body secretions can be readily loosened and removed.

Skin cleansing is quite a complex process. An ideal cleanser should remove dirt effectively without causing irritation or damage to the skin. Surfactants enable oily dirt, which normally does not dissolve in water, to become dispersible in water and rinsed away. Each detergent surfactant molecule has a hydrophilic head that is attracted to water molecules and a hydrophobic tail that repels water and bonds itself to the oily dirt.

Surfactants can also be referred to as wetting agents and at least in some cases, foamers because they lower the surface tension of the medium in which they are dissolved. By lowering this interfacial tension between two media (e.g., skin/water) the surfactant plays a key role in the removal and suspension of dirt and oil. The lower surface tension of the water makes it easier to lift dirt and grease off the skin. As the hydrophilic head remains in the water, it pulls the dirt and oil that is bonded to the hydrophobic tail away from the skin and into the water.

That said, many surfactants can also have the harmful effects on the skin such as after-wash tightness, dryness, damage to the barrier function of the skin, redness, irritation and itching. It is believed that some surfactants irritate the skin by interacting with the skin's natural proteins, such as corneocytes. Corneocytes are protein complexes formed of threads of keratin in an organized matrix. The surfactants bind to these proteins allowing them to become over-hydrated, such that they swell. This swelling can make it easier for the cleanser ingredients to penetrate into the lower layers of the skin, possibly causing itching and irritation. Thus, personal care cleansing compositions that contain only eco-friendly, natural ingredients are highly desirable to minimize or eliminate these potential ill-effects primarily associated with synthetic cleanser ingredients.

Embodiments of the present invention preferably include mild, eco-friendly, natural surfactants. The detergent surfactants may include any suitable naturally-derived, naturally-processed anionic surfactant, non-ionic surfactant, cationic surfactant, amphoteric surfactant, lipoamino acid surfactant, or combinations thereof. In solution, anionic surfactants have a negatively charged head. They are particularly effective at oily soil cleaning, but they can react with the positively charged ions (e.g., calcium and magnesium) typically found in hard water, which can lead to partial deactivation.

Anionic surfactants suitable for use in embodiments of the present invention include cocoamphodipropionic acid, cocobetainamido amphopropionate, DEA-cocoamphodipropionate, disodium caproamphodiacetate, disodium caproamphodipropionate, disodium capryloamphodiacetate, disodium capryloamphodiproionate, disodium cocoamphocarboxyethylhydroxypropylsulfonate, disodium cocoamphodiacetate, disodium cocoamphodipropionate, disodium isostearoamphodiacetate, disodium isostearoamphodipropionate, disodium laureth-5 carboxyamphodiacetate, disodium lauroamphodiacetate, disodium lauroamphodipropionate, disodium oleoamphodipropionate, disodium PPG-2-isodeceth-7 carboxyamphodiacetate, disodium soyamphodiacetate, disodium stearoamphodiacetate, disodium tallowamphodiacetate, disodium wheatgermamphodiacetate, lauroamphodipropionic acid, quaternium-85, sodium arganamphoacetate, sodium babassuamphoacetate, sodium caproamphoacetate, sodium caproamphohydroxyproplylsulfonate, sodium amphopropionate, sodium capryloamphoacetate, sodium caproamphydroxypropylsulfonate, sodium caprylamphoacetate, sodium capryloamphohydroxyproplysulfonate, sodium capryloamphopropionate, sodium cocoamphoacetate, sodium cocoamphohydroxylproplysulfonate, sodium cocoamphopropionate, sodium cocoabutteramphoacetate, sodium cocoamphoacetate, sodium cocoamphohydroxypropylsulfonate, sodium cocoamphoacetate, sodiumcocoamphohydroxypropylsulfonate, sodium cocoamphopropionate, sodium cornamphopropionate, sodium cottonseedamphoacetate, sodium grapeseedamphoacetate, sodium hempseedamphoacetate, sodium isostearoamphoacetate, sodium isostearoamphopropionate, sodium lauroamphoacetate, sodium lauroamphydroxypropylsulfonate, sodium lauroampho PG-acetate phosphate, sodium lauroamphopropionate, sodium mangoamphoacetate, sodium myristoamphoacetate, sodium oleoamphoacetate, sodium oleoamphydroxypropylsulfonate, sodium oleoamphopropionate, sodium olivamphoacetate, sodium palmamphoacetate, sodium peanutamphoacetate, sodium ricebranamphoacetate, sodium ricebranamphoacetate, sodium rose hipsamphoacetate, sodium sesamphoacetate, sodium sheabutteramphoacetate, sodium stearoamphoacetate, sodium stearoamphohydroxypropylsulfonate, sodium stearoamphopropionate, sodium stearoamphopropionate, sodium sunflowerseedamphoacetate, sodium sweetalmondamphoacetate, sodium tallamphopropionate, sodium tallowamphoacetate, sodium undecylenoamphoacetate, sodium undecylenoamphoacetate, sodium wheat germamphoacetate, stearamidoethyl diethanolamine HCl, stearoamphoacetic acid, trisodium lauroampho PG-acetate chlorine phosphate.

Anionic surfactants may also include cocosulphate, anionic derivatives of alkylpolyglucosides, such as cocoglucoside and cocoglucoside cross-polymers. Coco-glucosides and their cross-polymers enable loose foaming and provide cleansing. Coco-glucosides and their cross-polymers may be derived from coconut oil, as well as other natural sources (e.g., fruit sugar and corn syrup). A preferred cocoglucoside cross-polymer is sodium bishydroxyethylglycinate cocoglucoside cross-polymer, which may be obtained from Colonial Chemical, Inc., South Pittsburg, Tenn. under the tradename POLYSUGA BETAINE C, which is derived from coconut oil and corn syrup.

An example of a cocoglucoside that may be suitable for use is available under the tradename LAMESOFT PO65 from Cognis Corporation, Ambler, Pa. LAMESOFT PO65 includes glyceryl oleate in addition to the cocoglucoside, which enhances moisturization. LAMESOFT PO65 is derived from coconut oil and fruit sugar.

Non-ionic surfactants do not have an electrically charged head, which makes them resistant to water hardness deactivation. They are excellent oil removers and are often used together with anionic surfactants because of their ability to contribute to making the surfactant system less hardness sensitive. Non-ionic surfactants that may be suitable for use in embodiments of the present invention include, for example, sucrose stearate, sucrose laurate, sucrose palmitate, lauryl glucoside, caprylyl/capryl glucoside, inulin lauryl carbamate, decyl glucoside, and combinations thereof. Decyl glucoside is produced by the reaction of glucose from corn starch with the fatty alcohol decanol which is derived from coconut. Decyl glucoside may be obtained from Cognis Corporation, Ambler, Pa.

Lipoamino acid surfactants provide an alternative to traditional surfactants. Lipoamino acids comprise a fatty acid component (lipophilic/hydrophobic portion) and an amino acid component (hydrophilic portion). The fatty acid component may be derived from natural sources such as coconut oil, palm oil, or similar sources. Various amino acid components may be used (e.g., glutamine, alanine, etc.). Lipoamino acids are highly biodegradable, and exhibit low toxicity, ecotoxicity and irritation effects. Foaming polypeptides are a form of lipoamino acid surfactants that are highly functional fatty acid derivatives of hydrolyzed proteins and amino acids. Foaming polypeptides are prepared by reacting hydrolyzed protein with a reactive fatty acid reagent. The fatty acid group is covalently bonded to the polypeptide chain as a result of the reaction. Once the carboxylic acid group is neutralized, a soluble foaming polypeptide is formed.

Lipoamino acid surfactants suitable for use in embodiments of the present invention include sodium cocoyl hydrolyzed wheat protein and sodium cocyl hydrolyzed soy protein (FOAM-SOY C) available from Arch Personal Care Products, South Plainfield, N.J. Lipoamino acid surfactants provide mild cleansing and a rich lather. Other suitable lipoamino acid surfactants include sodium cocoyl glutamate and disodium cocoyl glutamate (PERLASTAN SC 25 NKW or AMISOFT CS-22) available from Schill & Seilacher GmbH (Hamburg, Germany) and Ajinomoto North America (Fort Lee, N.J.), respectively. Another suitable lipoamino acid surfactant is sodium cocoyl alaninate, available under the tradename AMILITE ACS-12 from Ajinomoto North America.

In one embodiment, any included lipoamino acid surfactants are the only amine components included within the composition, such that harsh synthetic amine components are not included within the personal care cleansing composition. In other words, where surfactants including an amine group are included, such surfactants may be derived from natural amino acids, polypeptides, or combinations thereof (e.g., lipoamino acid surfactants). Thus, while the detergent surfactant components may include an amine group, harsh synthetic amine surfactants (e.g., ethanolamines) are not employed (e.g., DEAs (diethanolamines), MEAs (monoethanolamines), TEAs (triethanolamines).

In some embodiments, the detergent surfactants included within the personal care cleansing composition may include a sulfate group. Sodium lauryl sulfate and sodium laureth sulfate have long been used as synthetic sulfate surfactants, although these components are suspected to pose health risks. As a result, such components are preferably not included within the personal care cleansing compositions, although less processed sulfate alternatives such as sodium coco-sulfate may be employed as detergent surfactants. That said, sodium coco-sulfate is formed by sulfination of coconut oil fatty acids, which includes a blend of fatty acids typically including from about 8 to about 20 carbon atoms, while the lauryl group designates a group having 12 carbon atoms. Thus, sodium lauryl sulfate is a purified component of what may be found in sodium coco-sulfate. As such, if the personal cleansing composition is to be naturally-derived and naturally-processed, then no such sulfate components should be included.

The detergent surfactants are preferably included at a combined concentration in a range of about 10% to about 75% by weight of the composition, from about 15% to about 60% by weight of the composition, or from about 30% to about 50% by weight of the composition (e.g., about 40% by weight).

F. Other Ingredients

Embodiments of the present invention may include other optional ingredients including, but not limited to, preservatives, emollients, vitamins, plant extracts, exfoliants, fragrances, colorants, royal jelly (a honey bee secretion), and combinations thereof.

Embodiments of the present invention can be shelf stable using a naturally-derived and naturally-processed preservative systems for at least about six months, more preferably at least about one year and most preferably at least about three years. One suitable preservative system comprises enzyme-based oxygen scavenging antioxidant systems as described in U.S. Pat. No. 5,972,355 which is hereby incorporated by reference in its entirety. The combination of glucose, glucose oxidase and lactoperoxidase is one example of an enzyme-based oxygen scavenging antioxidant system. The combination of glucose, glucose oxidase and lactoperoxidase is available as BIOVERT from Arch Personal Care Products, South Planfield, N.J. The total of all components in a preservative system may typically be present in amounts ranging from about 0.1% to about 5% or from about 0.25% to about 1.5% by weight of the composition.

Another suitable natural preservative system comprises water, glycerin, citric acid, lactic acid, and ascorbic acid available as Jeeplex NAS from Jeen International Corp that can be used alone or in combination with potassium sorbate available from Jeen Chemical. Embodiments of the present invention can contain Jeeplex NAS in amounts ranging from about 0.1% to about 1% by weight and potassium sorbate in amounts ranging from about 0% to about 1% by weight of the composition. Preferred ranges are about 0.3% to about 0.7% Jeeplex NAS and about 0.2% to about 0.5% potassium sorbate.

Phenoxyethanol is a chemical preservative which is commonly used in natural personal care products. Embodiments of the present invention can contain phenoxyethanol in amounts up to about 1% by weight of the composition.

Additionally, it is anticipated that new preservative systems will continue to be developed for use in natural personal care products. It is intended that the compositions disclosed herein could be modified to include any such developments.

Although not naturally-derived or naturally-processed, it is noted that synthetic preservatives such as methyl parabens, ethyl parabens, propyl parabens, butyl parabens, methylisothiazolinone, methylchloroisothazolinone, isobutyl parabens, and DMDM hydantoin, and combinations thereof are compatable with the novel thickening systems disclosed herein.

Exfoliants break down and remove keratinized cells that naturally build up on the skin's surface. Normal skin, or even skin functioning at peak performance can benefit from an exfoliant. Exfoliants help restore a healthy, translucent glow many consumers strive for. Suitable eco-friendly, natural exfoliants include fruit seeds and fibers, grain powders, nut/seed meal, oil/wax beads, sugar and jojoba beads.

Fragrances and essential oils are often added to personal care compositions in small amounts to provide an aromatically pleasing effect. Suitable eco-friendly, natural fragrances and essential oils include those generally known to one of skill in the art. Examples include, but are not limited to, citrus essential oils, floral essential oils, and combinations thereof.

### IV. Examples

The personal care cleansing compositions disclosed herein provide thickening characteristics similar to those of synthetic personal care cleansing compositions. As a result, the thickening systems disclosed herein provide for high performing, natural formulations with a minimum, if any, synthetic components.

Four personal care cleansing compositions containing embodiments of the thickener system of the present invention were prepared. Formulation A included about 50% by weight carrier water (not including water present as blended with other components), about 40% collectively of detergent surfactants (i.e., sodium bis-hydroxyethylglycinate cocoglucoside crosspolymer, disodium cocoyl glutamate, sodium cocoyl glutamate, and sodium cocoyl alaninate), and about 1.55% thickener system by weight. The 1.55% of the thickener system included 0.7% xanthan gum, 0.75% sodium lauroyl lactylate, and 0.1% lauryl lactyl lactate. The balance of the composition comprised a preservative system, fragrance botanical extracts, essential oils and the like. The pH of Formulation A ranged from about 5.5 to about 6.5 and the viscosity from about 2500-3300 cps. The composition provided a consistent thickness throughout, without the presence of gooey or gelatinous blobs, both of which were observed with similar compositions that were thickened with only a gum.

Formulation B included about 50% by weight carrier water (not including water present as blended with other components), about 40% collectively of detergent surfactants (i.e., sodium bis-hydroxyethylglycinate cocoglucoside crosspolymer, disodium cocoyl glutamate, sodium cocoyl glutamate, and sodium cocoyl alaninate), and about 2.05% thickener system by weight. The 2.05% of the thickener system included 0.7% xanthan gum, 1.1% sodium lauroyl lactylate, and 0.25% lauryl lactyl lactate. The balance of the composition comprised a preservative system, fragrance botanical extracts, essential oils and the like. The pH of Formulation B ranged from about 5.5 to about 6.5 and the viscosity from about 2500-3300 cps. The composition provided a consistent thickness throughout, without the presence of gooey or gelatinous blobs, both of which were observed with similar compositions that were thickened with only a gum.

Formulation C included about 50% by weight carrier water (not including water present as blended with other components), about 40% collectively of detergent surfactants (i.e., sodium bis-hydroxyethylglycinate cocoglucoside crosspolymer, disodium cocoyl glutamate, sodium cocoyl glutamate, and sodium cocoyl alaninate), and about 1.6% thickener system by weight. The 1.6% of the thickener system included 0.65% xanthan gum, 0.85% sodium lauroyl lactylate, and 0.1% lauryl lactyl lactate. The balance of the composition comprised a preservative system, fragrance botanical extracts, essential oils and the like. The pH of Formulation C ranged from about 5.5 to about 6.5 and the viscosity from about 2500-3300 cps. The composition provided a consistent thickness throughout, without the presence of gooey or gelatinous blobs, both of which were observed with similar compositions that were thickened with only a gum.

Formulation D included a coco sulfate surfactant component, while formulations A-C were free of sulfate surfactants. Formulation D included about 72% by weight carrier water (not including water present as blended with other components), about 17% collectively of detergent surfactants (i.e., sodium bis-hydroxyethyleglycinate cocoglucoside crosspolymer, disodium cocoyl glutamate, sodium cocoyl glutamate, and sodium coco-sulfate), and about 1.3% thickener system by weight. The 1.3% of the thickener system included 0.1% xanthan gum, 0.7% sodium lauroyl lactylate, and 0.5% lauryl lactyl lactate. The balance of the composition comprised a preservative system, fragrance botanical extracts, essential oils and the like. The pH of Formulation D ranged from about 5.5 to about 6.5 and the viscosity from about 2500-3300 cps. The composition provided a consistent thickness throughout, without the presence of gooey or gelatinous blobs, both of which were observed with similar compositions that were thickened with only a gum.

For comparative purposes, Formulation E was prepared without the novel thickening system. Accordingly, the composition included about 48% by weigh carrier water (not including water present as blended with other components), about 25% collectively of detergent surfactants (ie., decyl glycoside, coco-glucoside, lauryl glucoside, betaine, coco-betaine, caprylyl/capryl grlucoside), and 0.8% thickener system be weight. The 0.8% thickener system included 0.3% xanthan gum and 0.5% sodium stearoyl lactylate. The balance of the composition comprised a preservative system, fragrance botanical extracts, essential oils and the like. The pH of Formulation E ranged from about 6.0 to about 6.5 and the viscosity was about 700 cps. The composition had an unpleasant consistency, which was gooey and included gelatinous blobs.

While embodiments of the present invention has been described with reference to what are presently considered to be the preferred embodiments, it is to be understood that the invention is not limited to these embodiments. To the contrary, the invention is intended to cover various modifications and equivalent arrangements included within the spirit and scope of the appended claims.

We claim:

1. A liquid personal care cleansing composition comprising:
a carrier;
a detergent surfactant for personal cleansing;
a thickening system comprising;
a gum;
a lactate; and
a lactylate;
wherein the cleansing composition is free of ethanolamine thickener components.

2. A liquid personal care cleansing composition as recited in claim 1, wherein the carrier is aqueous.

3. A liquid personal care cleansing composition as recited in claim 1, wherein the gum is selected from the group consisting of xanthan gum, carrageenan, and gum arabic and the thickening system includes components that are naturally-derived and naturally-processed.

4. A liquid personal care cleansing composition as recited in claim 1, wherein the gum is present in a range of about 0.01% to about 2% by weight of the liquid personal care cleansing composition.

5. A liquid personal care cleansing composition as recited in claim 1, wherein the lactate is present in a range of about 0.01% to about 5% by weight of the liquid personal care cleansing composition.

6. A liquid personal care cleansing composition as recited in claim 1, wherein the lactylate is present in a range of about 0.01% to about 5% by weight of the liquid personal care cleansing composition.

7. A liquid personal care cleansing composition as recited in claim 1, wherein a weight ratio of the gum to the lactylate is from about 0.1:1 to about 2:1.

8. A liquid personal care cleansing composition as recited in claim 1, wherein a weight ratio of the gum to the lactylate is from about 0.5:1 to about 1:1.

9. A liquid personal care cleansing composition as recited in claim 1, wherein a weight ratio of the lactylate to the lactate is from about 0.05:1 to about 0.75:1.

10. A liquid personal care cleansing composition as recited in claim 1, wherein a weight ratio of the lactylate to the lactate is from about 0.1:1 to about 0.25:1.

11. A liquid personal care cleansing composition as recited in claim 1, wherein the one or more detergent surfactants are present in a range of about 10% to about 75% by weight of the liquid personal care cleansing composition.

12. A liquid personal care cleansing composition as recited in claim 1, wherein the one or more detergent surfactants are present in a range of about 30% to about 50% by weight of the liquid personal care cleansing composition.

13. A liquid personal care cleansing composition as recited in claim 1, wherein the one or more detergent surfactants include a lipoamino acid surfactant.

14. A liquid personal care cleansing composition as recited in claim 1, wherein the cleansing composition is substantially free of sulfates.

15. An amide-free thickener system for use in a liquid personal care cleansing compositions, the thickener system comprising:
a gum;
a lactate; and
a lactylate;
wherein the thickener system is free of ethanolamines.

16. An amide-free thickener system as recited in claim 15, wherein the gum is selected from the group consisting of xanthan gum, carrageenan, and gum arabic and the thickening system includes components that are naturally-derived and naturally-processed.

17. An amide-free thickener system as recited in claim 15, wherein the gum is xanthan gum.

18. An amide-free thickener system as recited in claim 15, wherein the thickener system further comprises an inorganic salt.

19. A liquid personal care cleansing composition comprising:
a carrier;
a detergent surfactant for personal cleansing, the detergent surfactant including a lipoamino acid surfactant; and
a natural thickening system including components that are naturally-derived and naturally-processed, the natural thickening system comprising;
a gum present in a range of about 0.01% to about 2% by weight of the liquid personal care cleansing composition;
a lactate present in a range of about 0.05% to about 0.5% by weight of the liquid personal care cleansing composition; and
a lactylate present in a range of about 0.5% to about 1.5% by weight of the liquid personal care cleansing composition;
wherein the cleansing composition is free of ethanolamine thickener components.

20. The liquid personal care cleansing composition recited in claim 19, wherein the cleansing composition comprises at least 95% naturally-derived and naturally-processed components and is free of sulfates.
